(19) United States
(12) Patent Application Publication
(10) Pub. No.: US 2023/0170082 A1
(43) Pub. Date: Jun. 1, 2023

(54) METHOD OF PREDICTING WAITING DAYS FOR A MEDICAL EXAMINATION, APPARATUS FOR PREDICTING WAITING DAYS FOR A MEDICAL EXAMINATION, AND COMPUTER PROGRAM STORED ON RECORDING MEDIUM FOR EXECUTING THE METHOD

(71) Applicant: SAMSUNG LIFE PUBLIC WELFARE FOUNDATION, Seoul (KR)

(72) Inventors: Junghwan MOON, Seoul (KR); Keunhwa KIM, Seoul (KR); Choonbong SON, Seoul (KR); Byoungjun HWANG, Seoul (KR)

(73) Assignee: SAMSUNG LIFE PUBLIC WELFARE FOUNDATION, Seoul (KR)

(21) Appl. No.: 18/060,039

(22) Filed: Nov. 30, 2022

(30) Foreign Application Priority Data
Dec. 1, 2021 (KR) 10-2021-0170235

(51) Int. Cl. G16H 40/20; G16H 40/40

(52) U.S. Cl. CPC G16H 40/20; G16H 40/40

(57) ABSTRACT

Provided are a method of predicting waiting days for a medical examination requiring a reservation at a hospital, the method including acquiring examination prescription and execution data, calculating, based on the examination prescription and execution data, an average ratio of executed examinations for each day required from a prescription to the execution thereof, and an average number of prescriptions based on past weekdays, estimating, based on the average number of prescriptions based on past weekdays, an average number of prescriptions based on future weekdays for a predetermined period by using at least one time-series prediction model, calculating a monthly increment in the average number of prescriptions based on future weekdays, compared to the average number of prescriptions based on the past weekdays, and allocating, based on the average number of prescriptions based on the past weekdays and the monthly increment, an expected number of prescriptions based on future weekdays.

### CROSS-REFERENCE TO RELATED APPLICATION

This application is based on and claims priority under 35 U.S.C. § 119 to Korean Patent Application No. 10-2021-0170235, filed on Dec. 1, 2021, in the Korean Intellectual Property Office, the disclosure of which is incorporated by reference herein in its entirety.

### BACKGROUND

### 1. Field

One or more embodiments relate to a method of predicting waiting days for a medical examination, an apparatus for predicting waiting days for a medical examination, and a computer program stored on a recording medium to execute the method, and more particularly, to a method of predicting waiting days for a medical examination, in which waiting days for an examination that requires a reservation at a hospital may be efficiently predicted, an apparatus for predicting waiting days for a medical examination, and a computer program stored on a recording medium to execute the method.

### 2. Description of the Related Art

In line with the improved quality of life and the increased interest in health, the population using medical services is gradually increasing, and there has been a request for efficient management of reservations of medical services such as diagnosis, treatment, operations conducted by clinics and hospitals. To minimize the waiting from the standpoint of individuals, and for efficient resource management on the side of clinics and hospitals, it has now become common to manage the examination schedule through reservations at clinics and hospitals.

However, at the current point of reservation, only days to wait for until the examination is provided, but the expected waiting days at the future time point is not provided, and thus, it is difficult for individuals to manage the schedule, and at the end of the hospitals and clinics, it is difficult to set up suitable investment and operation plan for the examination equipment in advance, which delays the examinations.

### SUMMARY

One or more embodiments include a method of predicting waiting days for a medical examination, in which waiting days for an examination that requires a reservation at a hospital may be efficiently predicted, an apparatus for predicting waiting days for a medical examination, and a computer program stored on a recording medium to execute the method. However, the above objectives of the disclosure are exemplary, and the scope of the disclosure is not limited by the above objectives.

Additional aspects will be set forth in part in the description which follows and, in part, will be apparent from the description, or may be learned by practice of the presented embodiments of the disclosure.

According to one or more embodiments, a method of predicting waiting days for a medical examination requiring a reservation at a hospital, includes acquiring examination prescription (where a ‘prescription’ means an effective prescription, for which a reservation is made or an execution is conducted after the prescription, except for repeated prescriptions) and execution data, calculating, based on the examination prescription and execution data, an average ratio of executed examinations for each day required from a prescription to the execution thereof, and an average number of prescriptions based on past weekdays (where a ‘weekday’ means a working day), estimating, based on the average number of prescriptions based on past weekdays, an average number of prescriptions based on future weekdays for a predetermined period by using at least one time-series prediction model, calculating a monthly increment in the average number of prescriptions based on future weekdays, compared to the average number of prescriptions based on the past weekdays, and allocating, based on the average number of prescriptions based on the past weekdays and the monthly increment, an expected number of prescriptions based on future weekdays, and estimating waiting days for an examination for each future date based on the expected number of prescriptions,

The acquiring of the examination prescription and execution data may include distinguishing prescription departments by reflecting a difference in examination prescription and execution patterns between weekdays and weekends, and acquiring prescription data of prescriptions of the prescription departments for a predetermined period, prescription data of prescriptions, for which examinations are conducted after the prescriptions, and execution data.

The calculating of the average ratio of executed examinations for each day required from a prescription to the execution thereof, and the average number of prescriptions based on past weekdays may include calculating a number of prescriptions by date with respect to each prescription department based on the execution data, calculating an average ratio of executed examinations for each day required from a prescription to the execution thereof, based on the prescription data and the number of prescriptions by date, and calculating the average number of prescriptions based on past weekdays, by dividing the number of prescriptions for a predetermined period, by the number of weekdays

The estimating of the average number of prescriptions based on future weekdays may include estimating the average number of prescriptions based on future weekdays by using an ensemble model which combines result values of a plurality of time-series prediction models.

The estimating of waiting days for an examination for each future date may include setting an additional capacity that is additionally operable in addition to the capacity, when a date of examination requiring a reservation is a weekday, and when the estimated number of reservations for the date of examination exceeds a sum of the capacity and the additional capacity, distributing the excess capacity to a date prior to the date of examination according to a predetermined condition.

The estimating of the waiting days for an examination for each future date may include calculating, as waiting days for each future date, a section until a first day when two consecutive weekdays start to appear, on which the number of reservations compared to the capacity for each future date is less than or equal to a predetermined ratio.

According to one or more embodiment, an apparatus for predicting waiting days for a medical examination requiring a reservation at a hospital, includes a processor configured to acquire examination prescription and execution data, calculate, based on the examination prescription and execution data, an average ratio of executed examinations for each day required from a prescription to the execution thereof, and an average number of prescriptions based on past weekdays, estimate, based on the average number of prescriptions based on past weekdays, an average number of prescriptions based on future weekdays for a predetermined period by using at least one time-series prediction model, calculate a monthly increment in the average number of prescriptions based on future weekdays, compared to the average number of prescriptions based on the past weekdays, and allocate, based on the average number of prescriptions based on the past weekdays and the monthly increment, an expected number of prescriptions based on future weekdays, and estimate waiting days for an examination for each future date based on the expected number of prescriptions.

The processor may be further configured to distinguish prescription departments by reflecting a difference in examination prescription and execution patterns between weekdays and weekends, and acquire prescription data of prescriptions of the prescription departments for a predetermined period, prescription data of prescriptions, for which examinations are conducted after the prescriptions, and execution data.

The processor may be further configured to calculate a number of prescriptions by date with respect to each prescription department based on the execution data, calculate an average ratio of executed examinations for each day required from a prescription to the execution thereof, based on the prescription data and the number of prescriptions by date, and calculate the average number of prescriptions based on past weekdays, by dividing the number of prescriptions for a predetermined period, by the number of weekdays.

The processor may be further configured to estimate the average number of prescriptions based on future weekdays by using an ensemble model which combines result values of a plurality of time-series prediction models.

The processor may be further configured to set an additional capacity that is additionally operable in addition to the capacity, when a date of examination requiring a reservation is a weekday, and when the estimated number of reservations for the date of examination exceeds a sum of the capacity and the additional capacity, to distribute the excess capacity to a date prior to the date of examination according to a predetermined condition.

The processor may be further configured to calculate, as waiting days for each future date, a section until a first day when two consecutive weekdays start to appear, on which the number of reservations compared to the capacity for each future date is less than or equal to a predetermined ratio.

According to one or more embodiments, a computer program stored on a recording medium for executing the method above by using a computer is provided.

In addition to the aforesaid details, other aspects, features, and advantages will be clarified from the following drawings, claims, and detailed description.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a diagram for describing the structure and operation of an apparatus for predicting waiting days for a medical examination, according to an embodiment;

FIG. 2 is a diagram for describing a structure of a processor of the apparatus for predicting waiting days for a medical examination, according to an embodiment;

FIG. 3 is a flowchart of a method of predicting waiting days for a medical examination, according to an embodiment;

FIG. 4 is a flowchart of a method of predicting waiting days for a medical examination, according to another embodiment; and

FIGS. 5 to 8 are diagrams for describing a method of predicting waiting days for a medical examination, according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

As the disclosure allows for various changes and numerous embodiments, particular embodiments will be illustrated in the drawings and described in detail in the description. The effects and features of the disclosure, and ways to achieve them will become apparent by referring to embodiments that will be described later in detail with reference to the drawings. However, the disclosure is not limited to the following embodiments but may be embodied in various forms.

Hereinafter, embodiments will be described in detail with reference to the accompanying drawings, and in the description with reference to the drawings, like reference numerals denote like elements, and redundant description thereof will be omitted.

While such terms as “first” or “second,” etc., may be used to describe various elements, such elements must not be limited to the above terms. The above terms are used only to distinguish one element from another. Also, an expression used in the singular form encompasses the expression in the plural form, unless it has a clearly different meaning in the context. Also, it will be further understood that the terms “comprise” and/or “have” used herein specify the presence of stated features or elements, but do not exclude the presence or addition of one or more other features or elements.

In the drawings, for convenience of description, sizes of elements may be exaggerated or contracted. For example, since sizes and thicknesses of elements in the drawings are arbitrarily illustrated for convenience of explanation, the disclosure is not limited thereto.

In the embodiments below, it will be understood when a portion such as an area, an element, a unit, a block or a module is referred to as being “on” or “above” another portion, it can be directly on or above the other portion, or intervening portion may also be present. It will also be understood that when an area, an element, a unit, a block or a module is referred to as being “connected to” another area, element, unit, block or module, it can be directly connected to the another element, or it can be indirectly connected to the another element with another area, element, unit, block or module included therebetween.

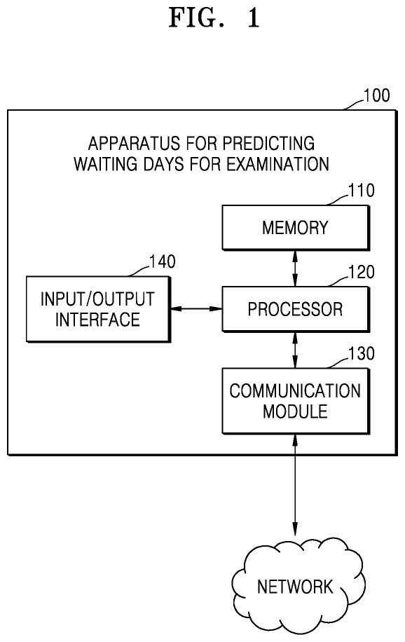

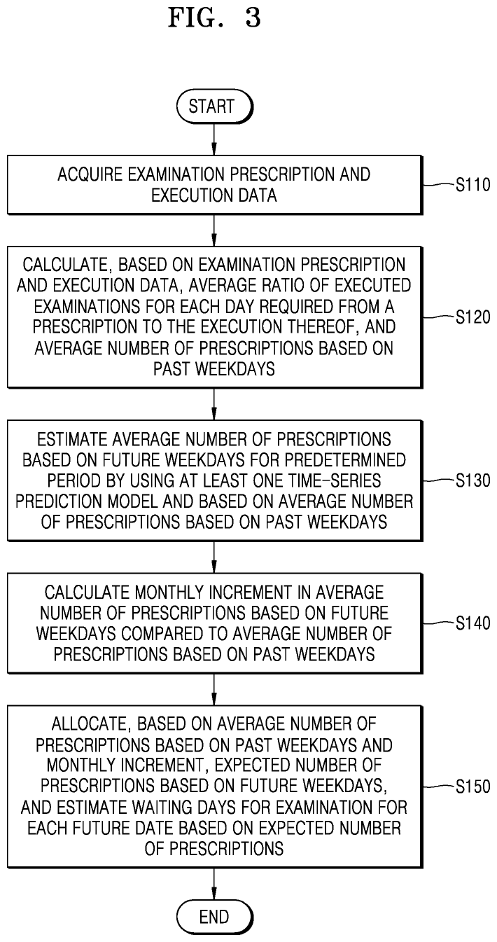

FIG. 1 is a diagram for describing the structure and operation of an apparatus for predicting waiting days for a medical examination, according to an embodiment. FIG. 2 is a diagram for describing a structure of a processor of the apparatus for predicting waiting days for a medical examination, according to an embodiment.

First, referring to FIG. 1, the apparatus 100 for predicting waiting days for a medical examination, according to an embodiment, may include a memory 110, a processor 120, a communication module 130, and an input/output interface 140. However, the disclosure is not limited thereto, and the apparatus 100 for predicting waiting days for a medical examination may further include other components, or some components may be omitted. Some components of the apparatus 100 for predicting waiting days for a medical examination may be divided into a plurality of devices, or a plurality of components may be merged into a single device.

The memory 110 may include a computer-readable recording medium, and may include a random access memory (RAM), a read-only memory (ROM), and a permanent mass storage device such as a disk drive. In addition, the memory 110 may temporarily or permanently store program code and a time-series prediction model for controlling the apparatus 100 for predicting waiting days for a medical examination. For example, the memory 110 may store examination prescription and execution data.

The processor 120 may acquire examination prescription and execution data. In addition, the processor 120 may calculate, based on the examination prescription and execution data, an average ratio of executed examinations for each day required from a prescription to the execution thereof and an average number of prescriptions based on past weekdays. In addition, the processor 120 may estimate, based on the average number of prescriptions based on past weekdays, an average number of prescriptions based on future weekdays for a predetermined period by using at least one time-series prediction model. Also, the processor 120 may calculate a monthly increment in the average number of prescriptions based on future weekdays compared to the average number of prescriptions based on past weekdays. Also, the processor 120 may allocate an expected number of prescriptions based on future weekdays based on the average number of prescriptions based on past weekdays and on the monthly increment. In addition, the processor 120 may estimate waiting days for an examination for each future date based on the expected number of prescriptions.

The communication module 130 may provide a function for communicating with an external server through a network. For example, a request generated by the processor 120 of the apparatus 100 for predicting waiting days for a medical examination, according to program code stored in a recording device such as the memory 110, may be transmitted to an external server through a network under the control of the communication module 130. Conversely, a control signal, a command, content, a file, etc. provided under the control of a processor of an external server may be received by the apparatus 100 for predicting waiting days for a medical examination, through the communication module 130 through a network. For example, a control signal or a command of an external server received through the communication module 130 may be transmitted to the processor 120 or the memory 110.

The communication method is not limited, and may include not only a communication method using a communication network that a network may include (e.g., a mobile communication network, wired Internet, wireless Internet, a broadcasting network) but also short-range wireless communication between devices. For example, the network may include any one of a personal area network (PAN), a local area network (LAN), a campus area network (CAN), a metropolitan area network (MAN), a wide area network (WAN), a broadband network (BBN), the Internet, etc. Further, the network may include, but is not limited to, any one or more of a network topology including a bus network, a star network, a ring network, a mesh network, a star-bus network, a tree or hierarchical network, and the like.

In addition, the communication module 130 may communicate with an external server through a network. Although the communication method is not limited, the network may be a short-range wireless network. For example, the network may include a Bluetooth, Bluetooth Low Energy (BLE), or Wi-Fi communication network.

In addition, the apparatus 100 for predicting waiting days for a medical examination, according to the disclosure, may include the input/output interface 140. The input/output interface 140 may include a unit for an interface with an input/output device. For example, the input device may include a device such as a keyboard or a mouse, and the output device may include a device such as a display for displaying a communication session of an application. As another example, the input/output interface 140 may include a unit for an interface with a device in which functions for input and output are integrated into one, such as a touch screen. As a specific example, when processing a command of a computer program loaded in the memory 110, the processor 120 of the apparatus 100 for predicting waiting days for a medical examination may display a service screen or content on a display through the input/output interface 140.

In addition, in other embodiments, the apparatus 100 for predicting waiting days for a medical examination may include more components than those of FIG. 1. For example, the apparatus 100 for predicting waiting days for a medical examination may be implemented to include at least some of the above-described input/output devices, or may further include other components such as a battery and a charging device for supplying power to internal components, various sensors, and a database.

Hereinafter, the internal configuration of the processor 120 of the apparatus 100 for predicting waiting days for a medical examination, according to an embodiment, will be reviewed in detail with reference to FIG. 2. For better understanding, it is assumed that the processor 120 to be described below is the processor 120 of the apparatus 100 for predicting waiting days for a medical examination, illustrated in FIG. 1.

The processor 120 of the apparatus 100 for predicting waiting days for a medical examination may include a data acquisition unit 121, a past weekdays-basis average number of prescriptions calculation unit 122, a future weekdays-basis average number of prescriptions calculation unit 123, a monthly increment calculation unit 124, and an examination-waiting days estimation unit 125. According to some embodiments, components of the processor 120 may be selectively included or excluded from the processor 120. In addition, according to some embodiments, the components of the processor 120 may be separated or combined to express the functions of the processor 120.

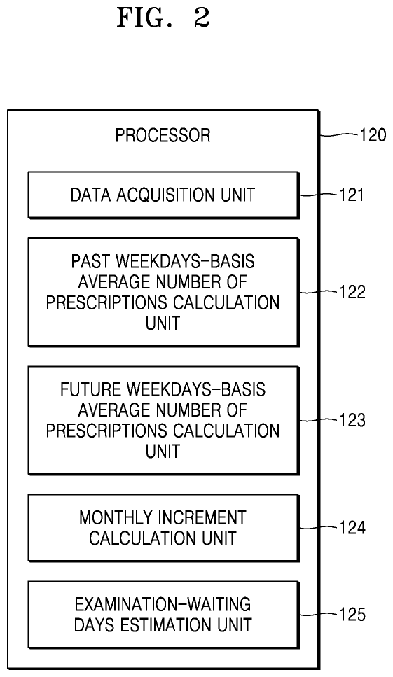

The processor 120 and the components of the processor 120 may control the apparatus 100 for predicting waiting days for a medical examination, so as to perform the operations included in the method of predicting waiting days for a medical examination, of FIG. 3 (S110 to S150). For example, the processor 120 and the components of the processor 120 may be implemented to execute instructions according to code of an operating system included in the memory 110 and code of at least one program. Here, the components of the processor 120 may be expressions of different functions of the processor 120, which are performed by the processor 120 according to a command provided by program code stored in the apparatus 100 for predicting waiting days for a medical examination. The internal configuration and certain operation of the processor 120 will be described with reference to a flowchart of the method of predicting waiting days for a medical examination, of FIG. 3.

FIG. 3 is a flowchart of a method of predicting waiting days for a medical examination, according to an embodiment.

Referring to FIG. 3, in operation S110, the processor 120 may acquire examination prescription and execution data.

The processor 120 according to an embodiment may distinguish a prescription department by reflecting a difference in examination prescription and execution patterns between weekdays and weekends. In addition, the processor 120 may acquire prescription data of prescriptions of a prescription department for a predetermined period, prescription data of prescriptions, for which examinations are conducted after the prescriptions, and execution data.

For example, the processor 120 may distinguish between prescription departments to reflect a difference in the patterns between weekdays and weekends. For example, the prescription departments may be divided into hemato-oncology and others. Also, the processor 120 may acquire execution data (e.g., including dates of execution and dates of prescriptions) for a certain past period (e.g., one year). Also, the processor 120 may acquire prescription data for a certain past period (e.g., one year).

In operation S120, the processor 120 may calculate, based on the examination prescription and execution data, an average ratio of executed examinations for each day required from a prescription to the execution thereof and an average number of prescriptions based on past weekdays.

The processor 120 according to an embodiment may calculate the number of prescriptions by date for each prescription department based on the execution data. Also, the processor 120 may calculate, based on the prescription data and the number of prescriptions by date, an average ratio of executed examinations for each day required from a prescription to the execution thereof. Also, the processor 120 may calculate an average number of prescriptions based on past weekdays, by dividing the number of prescriptions for a predetermined period by the number of weekdays.

For example, the processor 120 may calculate the number of cases per day, as to after how many days an examination is made after the date of prescription (e.g., 3 days) for each of prescription departments distinguished to reflect the pattern difference between weekdays and weekends (e.g., hemato-oncology and other departments). Subsequently, the processor 120 may calculate an average ratio of executed examinations for each day required from a prescription to the execution thereof, by dividing each number of cases by the total number of cases. For example, in order to increase the accuracy of a prediction model for waiting days for an examination for each future date, the processor 120 may limit data used when calculating the average ratio of executed examinations for each day required from a prescription to the execution thereof, to past data that is similar to the waiting days at the present time (e.g., 30 days). Also, the processor 120 may calculate the average number of prescriptions based on weekdays by dividing the number of prescriptions for a certain past period (e.g., 10 months) by the number of weekdays during the corresponding period.

In operation S130, the processor 120 may estimate an average number of prescriptions based on future weekdays for a predetermined period by using at least one time-series prediction model and based on the average number of prescriptions based on past weekdays.

The processor 120 according to an embodiment may estimate the average number of prescriptions based on future weekdays by using an ensemble model in which result values of a plurality of time-series prediction models are combined.

For example, the processor 120 may obtain an average number of prescriptions based on weekdays for each month for a certain past period (e.g., 7 years) by prescription departments divided to reflect the pattern difference between weekdays and weekends (e.g., hemato-oncology and other departments). Subsequently, the processor 120 may calculate an average number of prescriptions based on monthly weekdays for a certain future period (e.g., 9 months) through at least one time-series model. For example, the processor 120 may estimate the average number of prescriptions based on future weekdays by using an ensemble model that combines and utilizes a plurality of time-series models, in order to increase the accuracy of predicting the average number of prescriptions based on future weekdays.

In operation S140, the processor 120 may calculate a monthly increment in the average number of prescriptions based on future weekdays compared to the average number of prescriptions based on past weekdays.

For example, the processor 120 may derive an increment in the average number of prescriptions based on weekdays for each month for a certain future period (e.g., 9 months) (e.g., an expected increment in the number of prescriptions after 1 month compared to the past 10 months: 1.016794 (Hemato-oncology), 1.014449 (Others), an expected increment in the number of prescriptions after 2 months compared to the past 10 months: 1.016794 (Hemato-oncology), 1.005481 (Others), an expected increment in the number of prescriptions after 3 months compared to the past 10 months: 1.102290 (Hemato-oncology), 1.039362 (Others), etc.) compared to the average number of prescriptions based on weekdays for a certain period in the past (e.g., 10 months) to be used in calculation of waiting days.

In operation S150, the processor 120 may allocate, based on the average number of prescriptions based on the past weekdays and the monthly increment, an expected number of prescriptions based on future weekdays, and estimate waiting days for an examination for each future date based on the expected number of prescriptions.

The processor 120 according to an embodiment may set additional capacity that can be operated in addition to the capacity if the date of examination requiring a reservation is a weekday. In addition, when the estimated number of reservations for the date of examination exceeds a sum of the capacity and the additional capacity, the processor 120 may distribute the excess capacity to a date prior to the date of examination according to predetermined conditions.

The processor 120 according to an embodiment may calculate, as waiting days for each future date, a section until a first day when two consecutive weekdays start to appear, on which the number of reservations compared to the capacity for each future date is less than or equal to a predetermined ratio. For example, the processor may obtain reservation data at the present time and capacity data expected to be operated for a certain period in the future (e.g., 9 months) by date. In addition, the processor 120 may allocate the expected number of prescriptions for the corresponding month based on future weekdays. For example, the processor 120 may multiply the average number of prescriptions for a certain period in the past by an increment in the average number of prescriptions by month for a certain period in the future.

In addition, the processor 120 may update the waiting days for each future date by adding, to a reservation data value of each future date, a value obtained by multiplying the average number of prescriptions for a certain period in the past by a rate at which execution is to be made for each future date when a prescription is issued on the weekday. For example, the processor 120 may set the additional capacity that can be additionally operated on weekdays in addition to the capacity. In addition, when there are more reservations than the capacity on non-weekdays or more reservations than the sum of the capacity and the additional capacity on weekdays, the processor 120 may sequentially fill the excess reservations from earlier dates where the reservations are not yet full. In this case, the processor 120 may repeat the distribution operation until the excess reservations are eliminated, by distinguishing between a prescription department (e.g., hemato-oncology department) for which the excess reservations are distributed on any days where reservation is not full, regardless of whether it is a weekday, and a prescription department (e.g., other departments) for which the excess reservations are distributed only on weekdays where reservation is not full.

In addition, the processor 120 may calculate and output waiting days for each future date. For example, the processor 120 may calculate, as waiting days, a section until a first day when two consecutive weekdays start to appear, on which the number of reservations compared to the capacity is equal to or less than a certain standard (e.g., 95%) for each inquired future date. In addition, the processor 120 may visualize and express the expected number of waiting days by date until a certain future date (e.g., 9 months later) derived from the current time, as a graph or the like.

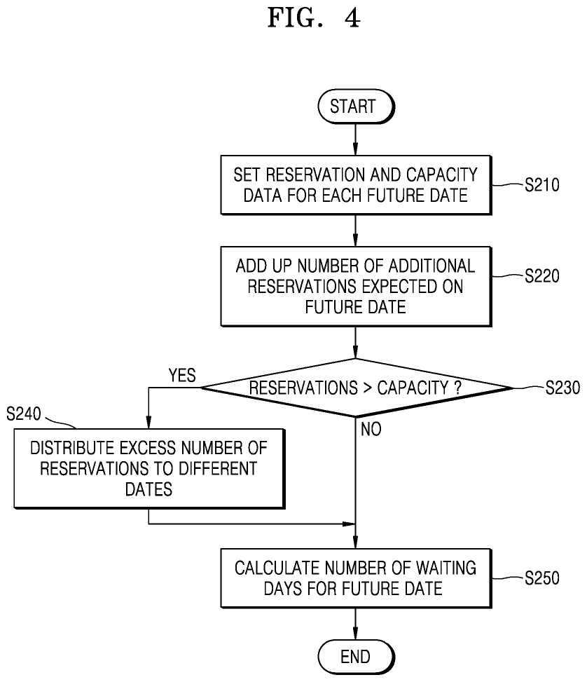

FIG. 4 is a flowchart of a method of predicting waiting days for a medical examination, according to another embodiment.

Referring to FIG. 4, in operation S210, the processor 120 may set reservation and capacity data for each future date. For example, the processor 120 may secure reservation and capacity data corresponding to a future date from the present time by distinguishing between weekdays or non-weekdays.

In operation S220, the processor 120 may add up the number of additional reservations expected on a future date. For example, the processor 120 may divide data into prescription departments so as to distinguish between weekday and weekend patterns, and calculate the number of executions and prescriptions by date. For example, the processor 120 may predict the number of prescriptions by month in the future by using a time-series pattern of the number of prescriptions. In addition, the processor 120 may calculate the number of additional reservations expected for a future date by multiplying the number of prescriptions predicted for each month by the execution rate by date. In addition, from among the number of excess reservations exceeded when the expected number of additional reservations is added to the number of existing reservations on future dates, the processor 120 may first add the expected number of additional reservations for a prescription department, to which the reservations may be distributed regardless of whether the days is a weekday or not, to the number of existing reservations, and then add the expected number of additional reservations for a prescription department, to which the reservations may be distributed only for weekdays.

In operations S230 and S240, the processor 120 may distribute the excess number of reservations to different dates. For example, for weekdays, the processor 120 may determine that the number of reservations is exceeded when the number of reservations is greater than the sum of the capacity and additional persons. For example, in the case of a prescription department for which the excess number of reservations can be distributed regardless of whether or not it is a weekday, the processor 120 may sequentially distribute the number of reservations that exceed due to the addition of the expected number of reservations to the number of existing reservations, from the earlier dates on which the number of reservations is not exceeded, regardless of whether it is a weekday or not. Also, in the case of a prescription department for which the excess number of reservations can be distributed only on weekdays, the processor 120 may sequentially distribute the number of reservations that exceed due to the addition of the existing number of reservations to the number of existing reservations, from the earlier dates on which the number of reservations is not exceeded only on weekdays. For example, the processor 120 may repeat the process of distributing the excess number of reservations until there is no more excess number of reservations.

In operation S250, the processor 120 may calculate the number of waiting days for the future date. For example, the processor 120 may calculate and display, as waiting days, a period until a first day on which two consecutive weekdays are repeated, on which the number of reservations for each inquired future date does not exceed a certain standard (e.g., 95%) compared to the capacity.

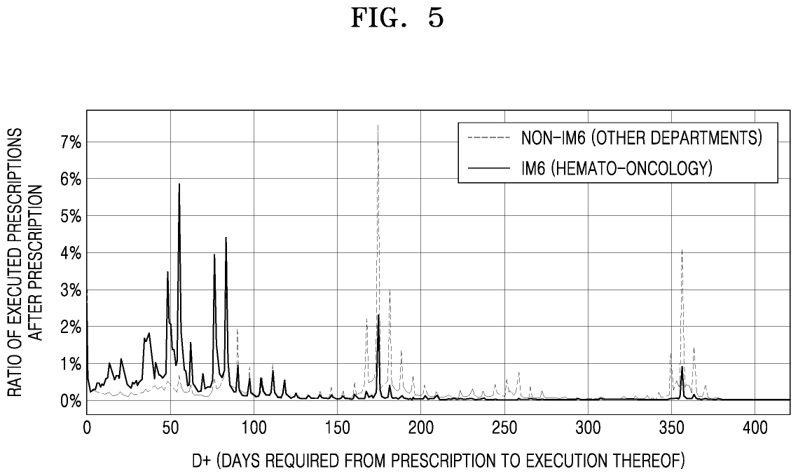

FIGS. 5 to 8 are diagrams for describing a method of predicting waiting days for a medical examination, according to an embodiment.

First, referring to FIG. 5, the number of prescriptions for a daily average CT scan examination based on weekdays from January to October 2021 is 541 cases for non-IM6 and 250 cases for IM6. Here, non-IM6 denotes other prescription departments except hemato-oncology, and IM6 denotes hemato-oncology. In addition, an increase rate of monthly prescriptions from November 2021 to July 2022 compared to January to October 2021 may be calculated.

Referring to FIG. 5, the distribution of the number of reservations to be additionally made from D+0 days to D+1448 days with respect to the day on which a prescription is issued may be calculated as follows.

Non-IM6: 541 cases×prescription increase rate for the corresponding month between November 2021 and July 2022 (e.g., November 2021: 1.014449)×distribution ratio of the number of executions from D+0 to D+1448 during January to October 2021.

IM6: 250 cases×prescription increase rate for the corresponding month between November 2021 and July 2022 (e.g., November 2021: 1.016794)×distribution ratio of the number of executions from D+0 to D+1448 during January to October 2021.

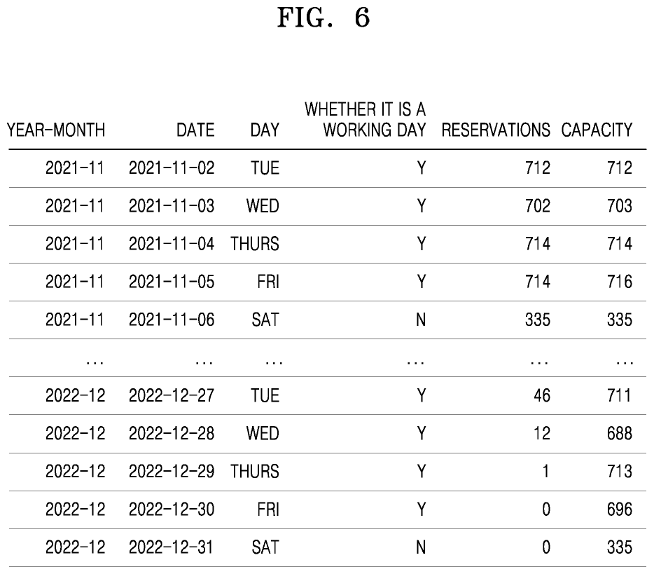

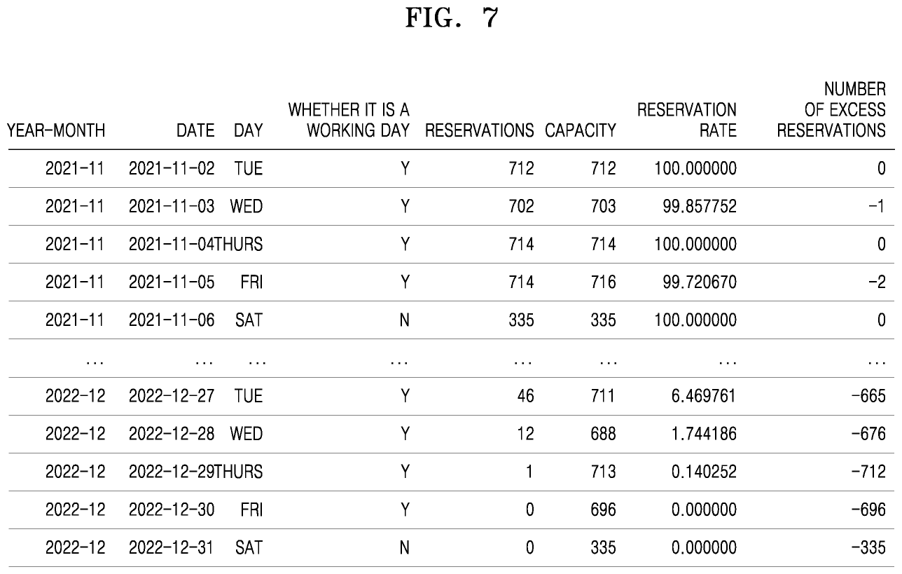

FIGS. 6 and 7 are diagrams for describing a method of calculating reservation data for calculating waiting days for each future date by reflecting the additional capacity, according to an embodiment.

First, referring to FIG. 6, a processor may add, to the inquired reservations on the day (e.g., Nov. 2, 2021) compared to the capacity, the number of reservations in consideration of prescriptions to be issued on weekdays from D+1 (e.g., Nov. 3, 2021). For example, the processor may set the available additional capacity to 10 in addition to the capacity. In addition, the processor may set the capacity in consideration of various situations. Also, the processor 120 may calculate a reservation rate (reservation/capacity) and excess reservations (reservation-capacity) by reflecting the set capacity. For example, the processor 120 may modify the additionally combined reservation information by reflecting the additional capacity setting. For example, referring to FIG. 7, when the item indicating whether the day is a working day is N (not applicable (N)), and the excess reservations are greater than 0, the processor 120 may sequentially move the excess reservations in order from a date where the excess reservations are less than 0. In addition, when the item indicating whether the day is a working day is Y (applicable (Y)), and the number of excess reservations is greater than the available additional capacity, the processor 120 may sequentially move the excess reservations exceeding the available additional capacity, in order from a date where the excess reservations are less than 0. For example, the processor 120 may fill reservations regardless of weekdays in the case of hemato-oncology, and fill reservations only if it is a weekday in case of non-hemato-oncology (other departments).

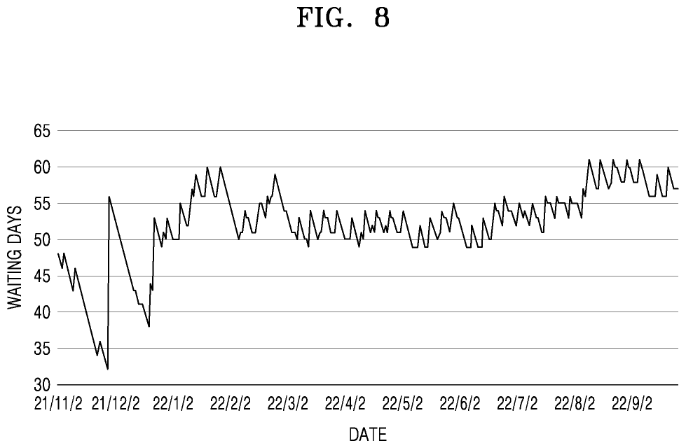

FIG. 8 is a diagram showing a result of prediction of waiting days for an examination, according to an embodiment. For example, FIG. 8 shows a result of prediction of waiting days for an examination, according to FIG. 7. In detail, FIG. 8 shows a result of estimating waiting days for an examination for each future date compared to the present time (Nov. 2, 2021).

The disclosure may be utilized when calculating waiting days for various examinations requiring a reservation at a hospital. When waiting days for various examinations may be accurately calculated from a future date, as according to the disclosure, factors that affect a delay in treatment may be minimized by securing appropriate investment and operation plans for examination equipment in advance. In addition, the disclosure may be applied effectively in hospitals where the demand from patients for examinations is greater than the available capacity of examinations. In addition, the disclosure may be applied to examinations, for which a prescription is issued on weekdays but which are conducted also on days that are other than weekdays.

In addition, according to the disclosure, waiting days from a future time may be provided compared to the method according to the related art, where only waiting days at the present time are provided. In addition, the pattern of the number of reservations to be filled in the future may be elaborately and accurately predicted.

In addition, according to the disclosure, the accuracy of predicting the number of waiting days for a future date may be improved. In addition, in order that a difference in practice patterns between weekdays and weekends can be distinguished, patient groups may be classified according to prescription departments and applied differently. In addition, a past execution pattern having a pattern similar to the aspect of the waiting days at the current time may be set to be utilized. In addition, a time-series ensemble model may be used to accurately predict the number of future prescriptions. In addition, in the case of weekdays, it is possible to increase the level of reality by reflecting the additional capacity that can be additionally operated, and the excess reservations exceed the capacity may be efficiently distributed according to prescription departments and whether it is a weekday or not.

In addition, according to the disclosure, as it becomes possible to predict waiting days not only for the present time but also for future dates, various operational attempts such as changing work scheduling may be made in the short term in order for hospitals to effectively manage waiting days, and in the long term, the predicted waiting days may be used in decision-making when deciding preemptive equipment investment. In addition, for patients, it is possible to schedule an examination in a timely manner.

The apparatus and/or system described above may be implemented as a hardware component, a software component, and/or a combination of hardware components and software components. Apparatuses and components described in the embodiments may be implemented using one or more general-purpose or special-purpose computers, for example, a processor, a controller, an arithmetic logic unit (ALU), a digital signal processor, a microcomputer, a field programmable gate array (FPGA), and a programmable logic unit (PLU), a microprocessor, or any other device capable of executing and responding to instructions. A processing device may run an operating system (OS) and one or more software applications running on the operating system. A processing device may also access, store, manipulate, process, and generate data in response to execution of software. For convenience of understanding, there are cases in which one processing device is used, but it will be obvious to those skilled in the art that a processing device may include a plurality of processing elements and/or a plurality of types of processing elements. For example, a processing device may include a plurality of processors or a single processor and a controller. Other processing configurations such as parallel processors are also possible.

Software may include computer program, code, instructions, or a combination of one or more of these, and may configure a processing device or independently or collectively configure give a command to the processing device to operate as desired. Software and/or data may be permanently or temporarily embodied in any tangible machine, a component, a physical device, virtual equipment, a computer storage medium or device, or in a transmitted signal wave in order to be interpreted by or provide instructions or data to a processing device. The software may be distributed on networked computer systems and stored or executed in a distributed manner. Software and data may be stored on one or more computer-readable recording media.

The method according to an embodiment may be embodied as program commands executable by various computer means and may be recorded on a computer-readable recording medium. The computer-readable recording medium may include program commands, data files, data structures, and the like separately or in combinations. The program commands recorded on the computer-readable recording medium may be specially designed and configured for the embodiments or may be well-known and be available to those of ordinary skill in the art of computer software. Examples of the computer-readable recording medium include magnetic media such as a hard disk, a floppy disk, or a magnetic tape, optical media such as a CD-ROM or a DVD, magneto-optical media such as a floptical disk, and a hardware device specially configured to store and execute program commands such as a ROM, a RAM, or a flash memory. Examples of the program commands include advanced language codes that may be executed by a computer by using an interpreter or the like as well as machine language codes made by a compiler. The hardware devices described above may be configured to operate as one or more software modules to perform the operations of the embodiments, and vice versa.

According to an embodiment as described above, a method for predicting waiting days for an examination that requires a reservation at a hospital, an apparatus for predicting waiting days for an examination, and a computer program stored in a recording medium to execute the method may be implemented. The scope of the disclosure, however, is not limited by these effects.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A method of predicting waiting days for a medical examination requiring a reservation at a hospital, the method comprising:
acquiring examination prescription and execution data;
calculating, based on the examination prescription and execution data, an average ratio of executed examinations for each day required from a prescription to the execution thereof, and an average number of prescriptions based on past weekdays;
estimating, based on the average number of prescriptions based on past weekdays, an average number of prescriptions based on future weekdays for a predetermined period by using at least one time-series prediction model;
calculating a monthly increment in the average number of prescriptions based on future weekdays, compared to the average number of prescriptions based on the past weekdays; and
allocating, based on the average number of prescriptions based on the past weekdays and the monthly increment, an expected number of prescriptions based on future weekdays, and estimating waiting days for an examination for each future date based on the expected number of prescriptions.

2. The method of claim 1, wherein the acquiring of the examination prescription and execution data comprises:
distinguishing prescription departments by reflecting a difference in examination prescription and execution patterns between weekdays and weekends; and
acquiring prescription data of prescriptions of the prescription departments for a predetermined period, prescription data of prescriptions, for which examinations are conducted after the prescriptions, and execution data.

3. The method of claim 2, wherein the calculating of the average ratio of executed examinations for each day required from a prescription to the execution thereof, and the average number of prescriptions based on past weekdays comprises:
calculating a number of prescriptions by date with respect to each prescription department based on the execution data;
calculating an average ratio of executed examinations for each day required from a prescription to the execution thereof, based on the prescription data and the number of prescriptions by date; and
calculating the average number of prescriptions based on past weekdays, by dividing the number of prescriptions for a predetermined period, by the number of weekdays.

4. The method of claim 3, wherein the estimating of the average number of prescriptions based on future weekdays comprises estimating the average number of prescriptions based on future weekdays by using an ensemble model which combines result values of a plurality of time-series prediction models.

5. The method of claim 1, wherein the estimating of waiting days for an examination for each future date comprises:
setting an additional capacity that is additionally operable in addition to the capacity, when a date of examination requiring a reservation is a weekday; and
when the estimated number of reservations for the date of examination exceeds a sum of the capacity and the additional capacity, distributing the excess capacity to a date prior to the date of examination according to a predetermined condition.

6. The method of claim 5, wherein the estimating of the waiting days for an examination for each future date comprises calculating, as waiting days for each future date, a section until a first day when two consecutive weekdays start to appear, on which the number of reservations compared to the capacity for each future date is less than or equal to a predetermined ratio.

7. An apparatus for predicting waiting days for a medical examination requiring a reservation at a hospital, the apparatus comprising
a processor configured to acquire examination prescription and execution data; calculate, based on the examination prescription and execution data, an average ratio of executed examinations for each day required from a prescription to the execution thereof, and an average number of prescriptions based on past weekdays; estimate, based on the average number of prescriptions based on past weekdays, an average number of prescriptions based on future weekdays for a predetermined period by using at least one time-series prediction model; calculate a monthly increment in the average number of prescriptions based on future weekdays, compared to the average number of prescriptions based on the past weekdays; and allocate, based on the average number of prescriptions based on the past weekdays and the monthly increment, an expected number of prescriptions based on future weekdays, and estimate waiting days for an examination for each future date based on the expected number of prescriptions.

8. The apparatus of claim 7, wherein the processor is further configured to distinguish prescription departments by reflecting a difference in examination prescription and execution patterns between weekdays and weekends; and acquire prescription data of prescriptions of the prescription departments for a predetermined period, prescription data of prescriptions, for which examinations are conducted after the prescriptions, and execution data.

9. The apparatus of claim 8, wherein the processor is further configured to calculate a number of prescriptions by date with respect to each prescription department based on the execution data, calculate an average ratio of executed examinations for each day required from a prescription to the execution thereof, based on the prescription data and the number of prescriptions by date, and calculate the average number of prescriptions based on past weekdays, by dividing the number of prescriptions for a predetermined period, by the number of weekdays.

10. The apparatus of claim 9, wherein the processor is further configured to estimate the average number of prescriptions based on future weekdays by using an ensemble model which combines result values of a plurality of time-series prediction models.

11. The apparatus of claim 7, wherein the processor is further configured to set an additional capacity that is additionally operable in addition to the capacity, when a date of examination requiring a reservation is a weekday, and when the estimated number of reservations for the date of examination exceeds a sum of the capacity and the additional capacity, to distribute the excess capacity to a date prior to the date of examination according to a predetermined condition.

12. The apparatus of claim 11, wherein the processor is further configured to calculate, as waiting days for each future date, a section until a first day when two consecutive weekdays start to appear, on which the number of reservations compared to the capacity for each future date is less than or equal to a predetermined ratio.

13. A computer program stored on a recording medium for executing the method of claim 1 by using a computer.
